(19) United States
(12) Patent Application Publication
(10) Pub. No.: US 2011/0238038 A1
(43) Pub. Date: Sep. 29, 2011

(54) INTRADERMAL MINI - NEEDLE INTERFACE AND ASSOCIATED DEVICES AND METHODS

(75) Inventors: Yoel Sefi, DN Merom Hagalil (IL); Yotam Levin, Nes Ziona (IL); Yehoshua Yeshurun, Haifa (IL); Nadav Agian, Kfar Yona (IL)

(73) Assignee: Nanopass Technologies Ltd., Nes Ziona (IL)

(21) Appl. No.: 13/132,930

(22) PCT Filed: Dec. 3, 2009
(86) PCT No.: PCT/IB09/55493

§ 371 Date: Jun. 6, 2011

Related U.S. Application Data
(60) Provisional application No. 61/119,747, filed on Dec. 4, 2008.

(51) Int. Cl. A61M 5/46

(52) U.S. Cl. 604/506; 604/117

(57) ABSTRACT

An intradermal mini-needle interface has a penetration limiter providing a skin contact edge and a hollow hypodermic needle having a beveled penetrating portion protruding forward beyond the skin contact edge by no more than 3 mm. At least one skin contact surface defines a skin contact plane parallel to, or at a shallow angle to, the needle axis. The skin contact plane intersects the skin contact edge substantially at a base of the penetrating portion. Preferably, the penetration limiter is asymmetric under rotation about the needle axis.

### FIELD AND BACKGROUND OF THE INVENTION

The present invention relates to systems and methods for the intradermal delivery of substances into, or sampling of fluids from, the skin of a mammal and, in particular, systems and methods employing mini needles (herein referred to “intradermal needles” of “ID Needles”) for such purposes.

Intradermal drug delivery is known to be advantageous for a range of different medications and treatments, such as immunization, immune-modulation, gene delivery, aesthetic medicine, dermatology, local anesthesia, allergy, hypersensitivity, cosmetics and diagnostics. Conventionally, intradermal drug delivery is performed by a skilled medical professional using a hypodermic needle positioned bevel-up at a shallow angle relative to the skin surface, also known as the “Mantoux method”. Care is required to achieve the correct depth of penetration to ensure successful injection within the dermal layers rather than subcutaneously or reemerging above the skin. In many cases, it has been reported that this method results in leakage or overly deep delivery that may compromise the diagnostic or therapeutic benefit of such an injection. The bevel-up needle orientation is needed in order to facilitate positive engagement of the needle with the skin surface at such shallow angles and is anyway the standard practice with any acute angle hypodermic needle insertion (including for example for venipuncture into deeper layers). A bevel-down approach has also been suggested, but is not widely in use due to slower injection time and difficulties in technique. The use of conventional hypodermic needles for intradermal delivery is known to be painful, since nerve endings in the dermal layer are typically severed by the relatively large needles used and the length that is usually required for insertion into the skin, safely, without leakage (usually over 0.5 cm length).

Further, it has been hypothesized that intra-epidermal delivery of drugs, such as vaccines, may have a further enhanced biological effect. Despite its promising prospects, this approach has been largely neglected to date since no injection delivery devices are available for such shallow application.

Much interest has been shown in development of drug delivery devices which do not require skilled operation, for example, for self-administration of drugs by patients. One approach is that of a “mini-needle” device with an actuator which selectively deploys or retracts the needle so as to penetrate to a limited depth within the dermal layers. Examples of such a device are commercially available from Becton, Dickinson & Co. (USA) and are described in U.S. Pat. Nos. 6,843,781, 6,776,776, 6,689,118, 6,569,143, 6,569,123 and 6,494,865, and in US Patent Application Publication Nos. 20080045900, 20080033395, 20080015521. The needle canula of such devices typically projects between 1 and 2 millimeters, thereby defining the depth of penetration of the delivery system. Since the already-reduced-length bevel of the needle tip itself has a length of at least about 0.4-0.8 mm, devices based on conventional needle structures of this type (i.e., a hollow metal cylinder with a beveled point) cannot readily be used for sealed fluid delivery to penetration depths less than 1 mm, and most likely are required to be in the length of approximately 1.5 mm.

In order to overcome skin elasticity or otherwise improve penetration, various designs of such limiters have been proposed, as referenced above. Other designs include attaching an adhesive to the skin and penetrating through the adhesive, such as for example in PY et al. (WO2004032989A2); others proposed a stabilizer apparatus, such as for example US 20070118077 to Clarke et al.

One inherent limitation of the proposed configurations listed above is that the insertion depth is at the verge of the subcutaneous space. This is due to the combination of needle size (approximately 1.5 mm) and perpendicular insertion approach. This results in a delivery that is questionably shallow enough to produce the required biological effect, especially if the delivered volume is relatively large (above 0.1 ml). It is thought to be more painful (because it transverses more nerve endings in the dermis), and is supposedly less effective for immunization (which is thought to require targeting of immune potent cells not only in the dermis but also in the epidermis and at the junction between these two layers) than a properly performed intradermal drug delivery. The limiters or adapters proposed in the prior art typically direct a perpendicular insertion angle, and circumscribe or surround the needle in such a manner that the limiter applies a downward force on the tissue into which the needle is inserted, as will be detailed below with reference to FIG. 16A.

There is therefore a need to provide an intradermal mini-needle interface which will facilitate reliable intradermal liquid delivery at shallow angles, controlled delivery depths, and reduced leakage. It would also be advantageous to provide an intradermal delivery configuration in which a mini-needle would be inserted shallowly into tissue which is not downwardly compressed by contact pressure of a limiter or adapter.

### SUMMARY OF THE INVENTION

The present invention is an intradermal mini-needle interface and associated devices and methods.

According to an embodiment of the present invention there is provided, an intradermal mini-needle interface comprising: (a) a penetration limiter providing a skin contact edge; (b) a hollow hypodermic needle having a beveled penetrating portion protruding forward beyond the skin contact edge by no more than 3 mm, the penetrating portion having a central needle axis; and (c) at least one skin contact surface defining a skin contact plane parallel to, or at a shallow angle to, the needle axis, the skin contact plane intersecting the skin contact edge substantially at a base of the penetrating portion.

According to a further optional embodiment of the present invention, the penetration limiter is asymmetric under rotation about the needle axis.

According to a further optional embodiment of the present invention, the penetration limiter is non-encompassing relative to the needle axis.

According to a further optional embodiment of the present invention, the penetration limiter is provided by a projecting element extending along one side of the hypodermic needle and spaced therefrom by no more than 2 mm.

According to a further optional embodiment of the present invention, there is also provided a fluid inlet in fluid communication with the hypodermic needle, the fluid inlet defining an inlet axis, wherein the skin contact plane is at an oblique angle to the inlet axis.

According to a further optional embodiment of the present invention, the needle axis is non-parallel to the inlet axis.

According to a further optional embodiment of the present invention, the hypodermic needle is deployed substantially parallel to, and adjacent to, the skin contact plane.

According to a further optional embodiment of the present invention, the penetration limiter and the at least one skin contact surface are provided by a unitary block of polymer material.

According to a further optional embodiment of the present invention, the hypodermic needle is part of a needle adapter having an exposed needle of length greater than 3 mm, and wherein the penetration limiter and the at least one skin contact surface are provided by a supplementary adapter configured for receiving the exposed needle.

According to a further optional embodiment of the present invention, the intradermal mini-needle interface is integrated as part of a syringe body.

According to a further optional embodiment of the present invention, there is also provided a fluid inlet in fluid communication with the hypodermic needle, the fluid inlet being configured for removable attachment to a syringe.

According to a further optional embodiment of the present invention, there is also provided a pressure impulse supply arrangement associated with the hypodermic needle and deployed to deliver a high energy fluid jet through the hypodermic needle.

There is also provided according to a further embodiment of the present invention, a method for transferring a liquid through at least one layer of the skin by use of an intradermal mini-needle interface including: (a) a penetration limiter providing a skin contact edge; (b) a hypodermic needle having a beveled penetrating portion protruding forward beyond the skin contact edge by no more than 3 mm, the penetrating portion having a central needle axis; and (c) at least one skin contact surface defining a skin contact plane parallel to, or at a shallow angle to, the needle axis, the skin contact plane intersecting the skin contact edge substantially at a base of the penetrating portion, the method comprising the steps of: (i) penetrating at least one layer of the skin with the hypodermic needle; (ii) bringing the hypodermic needle to a position with its central axis at a shallow angle to the initial local surface of the skin such that the hypodermic needle extends into a portion of the skin which is not overlaid by the penetration limiter; and (iii) transferring a liquid via the hypodermic needle through at least one layer of the skin.

There is also provided according to a further embodiment of the present invention, a method for introducing a liquid through at least one layer of the skin, the method comprising the steps of: (a) penetrating at least one layer of the skin with a hollow needle; (b) maintaining the hollow needle with its central axis at an angle of less than 45 degrees to the initial local surface of the skin; and (c) directing a high energy jet of the liquid along the hollow needle so as to cause directional penetration of the liquid through tissue beyond an end of the hollow needle.

According to a further optional embodiment of the present invention, the hollow needle is part of an intradermal mini-needle interface comprising: (a) a penetration limiter providing a skin contact edge; (b) a hypodermic needle having a beveled penetrating portion protruding forward beyond the skin contact edge by no more than 3 mm, the penetrating portion having a central needle axis; and (c) at least one skin contact surface defining a skin contact plane parallel to, or at a shallow angle to, the needle axis, the skin contact plane intersecting the skin contact edge substantially at a base of the penetrating portion.

### BRIEF DESCRIPTION OF THE DRAWINGS

The invention is herein described, by way of example only, with reference to the accompanying drawings, wherein:

FIG. 1A is an isometric view of an intradermal mini-needle interface, constructed and operative according to an embodiment of the present invention.

FIG. 1B is an exploded cross sectional side view of the device of FIG. 1A.

FIG. 2 is an isometric view of a variant of the device of FIG. 1 including an open sleeve for the needle and a stepped contact surface.

FIG. 3 is an isometric view of another variant of the device of FIG. 1, including multiple needles.

FIG. 4 is an isometric view of another variant of the device of FIG. 1, including rounded contact region.

FIG. 5 is a schematic side view of another variant of the device of FIG. 1, constructed with an obtuse angle at the intersection of a forward penetration limiting surface and a skin contact surface.

FIG. 6 is an isometric partially cut-away view of another variant of the device of FIG. 1 including a bent needle.

FIG. 7A is an exploded isometric view of another variant of the device of FIG. 1, including an adapter body, a needle canula and a cover.

FIG. 7B is an isometric cut-away view of the adapter body of FIG. 7A showing a fluid flow path.

FIG. 7C is an enlarged cross sectional view of the assembled adapter body and needle canula of the device of FIG. 7A.

FIG. 7D is an isometric view of the device of FIG. 7A illustrating an example of orientation marker.

FIG. 8A is an isometric view of another variant of the device of FIG. 1.

FIG. 8B is an isometric cut-away view of the device of FIG. 8A.

FIGS. 9A-9C are partially cut-away side views illustrating needle configurations with a downward-facing bevel, an upward-facing bevel, and a sideways-facing bevel, respectively.

FIGS. 10A-10E are schematic views taken along the central axis of the needle illustrating exemplary forms of penetrations limiter which are asymmetric under rotation.

FIG. 11A is an exploded isometric view of an intradermal mini-needle interface, constructed and operative according to an embodiment of the present invention, implemented as a standard hypodermic needle adapter in combination with a supplementary side-insertion adapter.

FIG. 11B is an enlarged isometric view of the contact surfaces of the side insertion adapter of FIG. 11A.

FIG. 11C is enlarged isometric view of the intradermal mini-needle interface of FIG. 11A when assembled.

FIG. 12A is an exploded isometric view of an intradermal mini-needle interface, constructed and operative according to an embodiment of the present invention, integrated as part of a syringe.

FIG. 12B is an axial cross-sectional view of the syringe of FIG. 12A when assembled.

FIG. 13A is an isometric view of an intradermal mini-needle interface, constructed and operative according to an embodiment of the present invention, with a rearward pointing septum needle for use with an injector device, prior to assembly.

FIG. 13B is an isometric view of the device of FIG. 7A in an assembled condition.

FIG. 14A is a schematic illustration of a side-insertion-based jet injection technique according to an embodiment of the present invention

FIG. 14B illustrates a conventional jet injection geometry, for comparison with FIG. 14A.

FIG. 14C is a schematic isometric view of a side-insertion-based jet injection device, which may be implemented with any of the intradermal mini-needle interfaces described herein.

FIG. 14D is an enlarged cross sectional view illustrating an exemplary embodiment for enhanced jet-injection flow generation for use in the device of FIG. 14C, shown with the needle removed.

FIG. 14E is an enlarged partial view of FIG. 14D showing the needle in its assembled position.

FIG. 15A is an isometric view of an intradermal mini-needle interface, constructed and operative according to an embodiment of the present invention, implemented as part of an infusion set.

FIGS. 15B-15D are a sequence of schematic side views illustrating deployment of the infusion set of FIG. 15A on the skin.

FIGS. 16A and 16B are schematic side views illustrating the effect of performing perpendicular approach injection employing a mini-needle interface with a symmetrical penetration limiter (prior art) and an asymmetric penetration limiter according to an embodiment of the present invention, respectively.

FIG. 17A is an isometric view of an intradermal mini-needle interface, constructed and operative according to an embodiment of the present invention, configured to allow shallow penetration over an extended length of the needle.

FIG. 17B is an enlarged schematic side view of the distal part of the interface of FIG. 17A.

FIG. 17C is a schematic side view of the interface of FIG. 17A after insertion into the skin.

### DESCRIPTION OF THE PREFERRED EMBODIMENTS

The present invention is an intradermal mini-needle interface and associated devices and methods.

The principles and operation of devices and methods according to the present invention may be better understood with reference to the drawings and the accompanying description.

Referring now to the drawings, a number of embodiments of the invention can be described generically, using reference numerals which will be maintained throughout the description, as follows. Generally speaking, an intradermal mini-needle interface according to an embodiment of the present invention includes a penetration limiter 16 providing a skin contact edge 16a, and a hollow hypodermic needle 12 having a beveled penetrating portion 12a protruding forward beyond skin contact edge 16a by no more than 3 mm. At least one skin contact surface 18 defines a skin contact plane 19 parallel to, or at a shallow angle to, a central axis 13 of needle 12. Skin contact plane 19 intersects skin contact edge 16a substantially at a base of penetrating portion 12a.

At this stage, it will be immediately apparent that such embodiments of the present invention offer distinct advantages over conventional devices available for intradermal injection, and intradermal fluid sampling. Specifically, the use of a needle with no more than 2 mm protruding beyond the skin contact edge allows reliable intradermal injection without the level of skill required using a conventional exposed hypodermic needle. Provision of skin contact surfaces defining a shallow-angle skin contact plane which intersects the edge of the penetration limiter substantially at the base of the penetration portion of the needle also facilitates correct alignment of the needle for effective intradermal injection. This geometry also helps to ensure that the tip of the needle extends into tissue which is not overlaid by the penetration limiter, thereby allowing delivery of liquid into uncompressed tissue. These and other advantages of the present invention will become clearer from the following description.

While the invention will be exemplified herein primarily in the context of devices and methods for intradermal injection, it should be noted that fluid sampling, for example, for diagnostic purposes such as glucose sensing, also falls within the scope of the present invention. Furthermore, in some cases, a sensor may be incorporated within the needle, or a similarly shaped dedicated sensor element may replace the needle, to perform sensing directly at or near the skin interface.

Before addressing the features of embodiments of the present invention in more detail, it will be useful to define certain terminology as used herein in the description and claims. Firstly, embodiments of the invention are referred to as an “intradermal mini-needle interface.” The phrase “mini-needle interface” is used herein to refer to any device which provides a needle-based interface with the skin for transferring a liquid through at least one layer of the skin in which the maximum perpendicular penetration depth of the needle is limited by a penetration limiter to no more than 5 mm, preferably no more than 3 mm, and most preferably no more than 2 mm. The interface may be integrated as part of liquid delivery device, such as a pre-filled syringe, or may be implemented as an adapter for attachment to another device.

The term “integrated” is used in this context to refer to an interface which is integrally formed or attached to other parts of the device so that it is not readily separable therefrom in a non-destructive manner. An “adapter” on the other hand is an interface which is configured for attachment to another device to provide required functionality. Thus, for example, a device with a luer connector for attachment to a syringe or other liquid supply line is referred to as an adapter. A conventional hypodermic needle integrated with a luer connector for use with a syringe is an example of a “needle adapter” while reference will be made in certain embodiments to a “supplementary adapter” which connects over a needle adapter.

The term “hypodermic needle” is used herein in the description and claims to refer to a hollow cylindrical needle with beveled penetrating tip, typically formed of metal. Such needles are commonly referred to as hypodermic needles, even when employed for intradermal use.

Reference is made to a central needle axis. This refers to the central axis of the cylindrical body of the needle, disregarding the bevel. In the case of a bent needle, the central needle axis is taken to the axis of the needle near its tip.

Reference is made to one or more skin contact surfaces which define a “skin contact plane.” The skin contact plane corresponds to the plane of a flat surface against which the skin contact surfaces would sit in close contact, ignoring any obstruction due to the needle tip. The individual skin contact surfaces do not necessarily have flat surfaces aligned with the plane. In the particular example of a single flat skin contact surface, the skin contact plane is coincident with the skin contact surface.

Reference is made herein to a “shallow angle”. The term “shallow angle” is used herein to refer to a direction which forms a sharp angle with the relevant reference plane, i.e., no more than 45 degrees, and most preferably, has an inclination of no more than 25 degrees thereto.

The penetration limiter of certain embodiments is referred to as being asymmetric under rotation. In this context, a shape is considered to be asymmetric under rotation if it is variant under all rotations other than multiples of 360 degrees.

Finally with regard to terminology, in certain embodiments, the needle is described as being parallel to and adjacent to the skin contact surface. In this context, “adjacent” is used to refer to a structure in which there is not a significant step felt between the level of the needle and the level of the skin contact surface. In quantitative terms, where described as adjacent, the closest side of the needle typically lies within about 2 needle diameters above or below the contact surface.

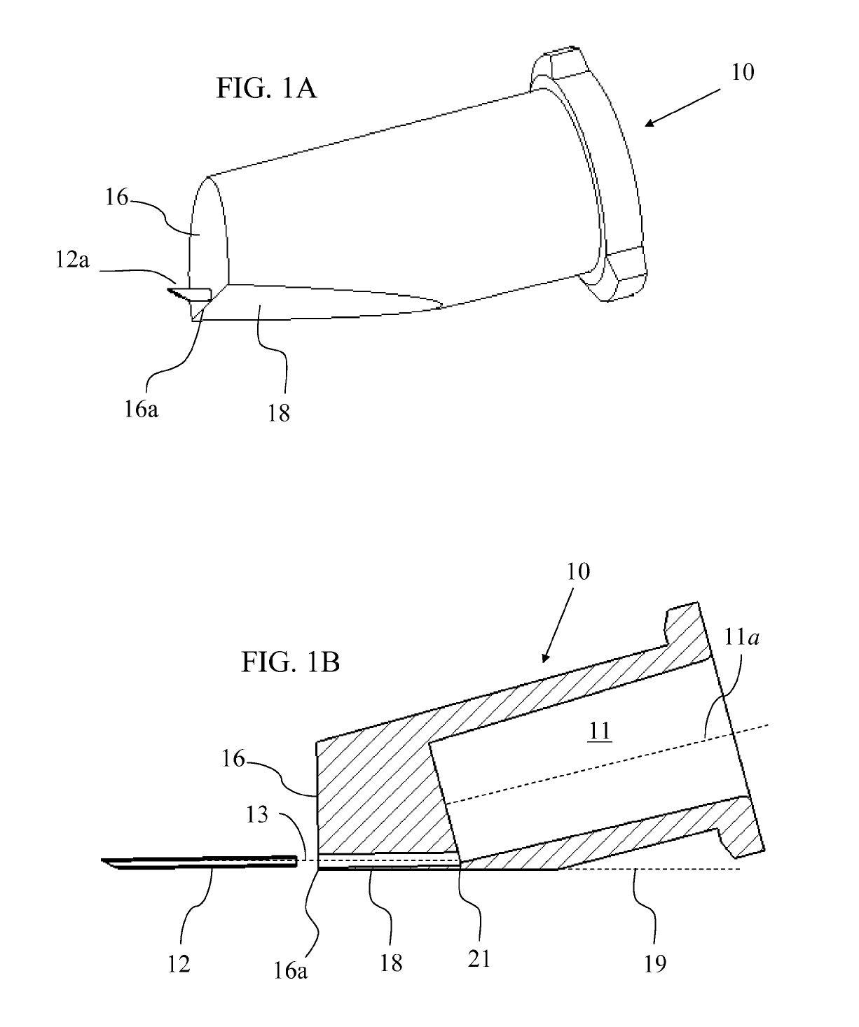

Turning now to FIGS. 1A and 1B, there is shown an intradermal mini-needle interface, generally designated 10, according to an embodiment of the present invention, including penetration limiter 16 with skin contact edge 16a, hypodermic needle 12 with beveled penetrating portion 12a as described generically above. In this case, skin contact plane 19 (FIG. 1B) coincides with the plane of a single flat skin contact surface 18.

In the case illustrated here, interface 10 is an adapter having a fluid inlet 11, in fluid communication with needle 12, and configured for removable attachment to a syringe or other standard delivery or sampling device. In the case illustrated here, inlet 11 is a standard luer connector with a central inlet axis 11a.

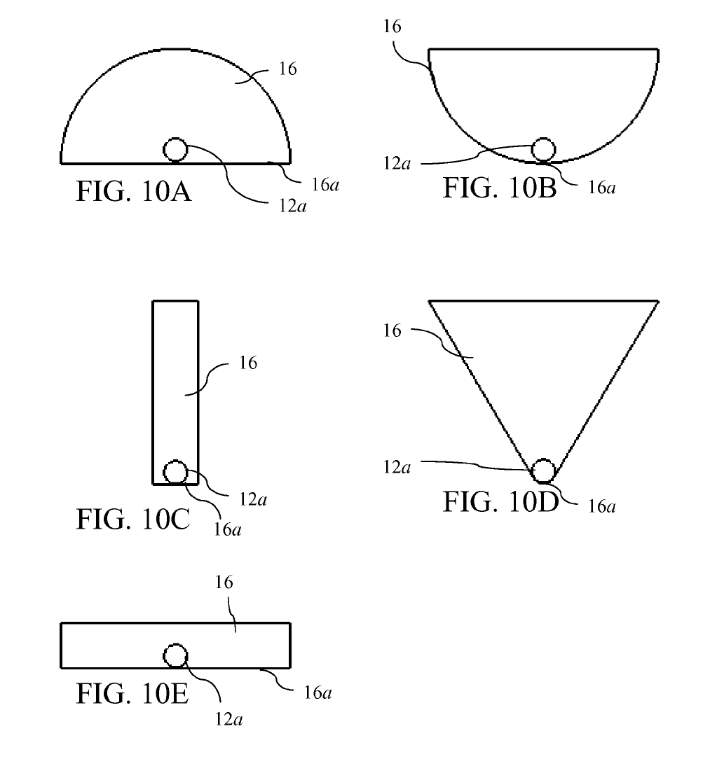

Penetration limiter 16 here is asymmetric under rotation about the needle axis, having an overall profile similar to that illustrated schematically in FIG. 10A.

Skin contact plane 19 is implemented here at an oblique, and preferably shallow, angle relative to inlet axis 11a. Needle axis 13 is here also non-parallel to inlet axis 11a. According to a particularly preferred feature of this embodiment, needle 12 is deployed substantially parallel to, and adjacent to, skin contact plane 19.

In this and various other preferred embodiments of the present invention, penetration limiter 16 and skin contact surface(s) 18 are advantageously provided by features of a unitary block of polymer material. The entire interface 10 is thus formed essentially from only two components, the polymer block and needle 12, which may be assembled and attached by various known methods, including but not limited to, application of adhesive or thermal welding of the polymer material. During assembly, the off-axis orientation of needle 12 relative to inlet 11 facilitates provision of a small step 21 against which needle 12 abuts to accurately define a fully inserted position of needle 12 for precise and reliable assembly. In certain cases, the needle may be introduced already during injection molding of the polymer block, with suitable precautions taken to ensure that it does not become blocked, as is know in the art.

Needles 12 suitable for implementing this and other embodiments of an interface according to the present invention are typically of outer diameter between about 100 and about 650 microns, and more preferably between about 200 and 400 microns. A short bevel is preferably used. The overall length of needle 12 is chosen according to the structure of the block of the interface, and may vary considerably. The exposed portion of the needle, corresponding to penetrating portion 12a, is preferably no more than 3 mm, and more preferably within the range of 1-2 mm.

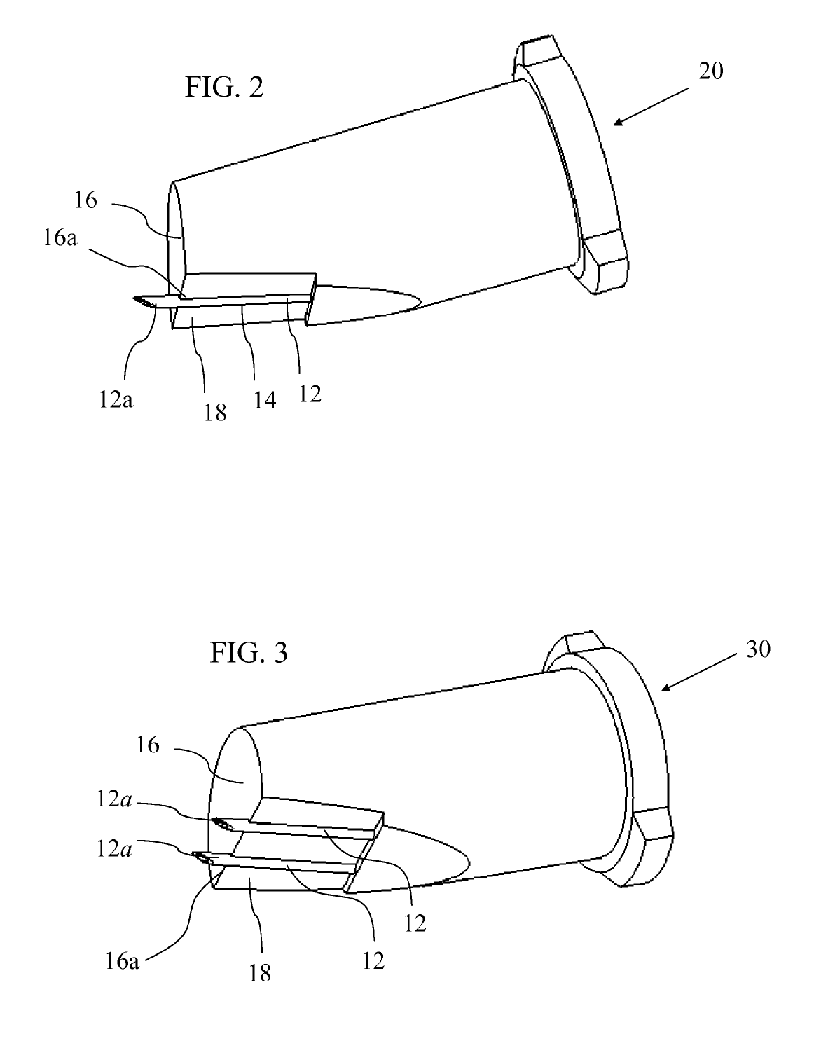

Turning now to FIG. 2, there is shown an intradermal mini-needle interface, generally designated 20, according to an alternative embodiment of the present invention. Interface 20 is generally structurally and functionally similar to interface 10, and similar reference numerals are used for similar elements. Interface 20 differs from interface 10 primarily in that at least part of the length of needle 12 is mounted in an open channel 14 so as to be exposed along at least part of contact surface 18. In the case shown here, contact surface 18 is subdivided into two surfaces with a step between them, thereby facilitating fluid connection to the rear end of needle 12, as will be further detailed in other embodiments below. The use of an open channel 14 to received needle 12 is believed to simplify assembly of the device, and sealing of the rear end of the needle by external application of adhesive around the point of entry of the needle into the polymer block. The stepped contact surface causes a small angular offset between the plane of each contact surface and the effective skin contact plane as defined above. However, the overall geometry and function of interface 20 remains very similar to that of interface 10.

Parenthetically, it should be noted here that the use of open-sided channel 14 here also changes the geometry of penetration limiter 16, resulting in a form which is non-encompassing relative to the needle axis. In other words, penetrating portion 12a of needle 12 is located at an extremity of the surface of penetration limiter 16 rather than being surrounded by material of the penetration limiter.

Turning now to FIG. 3, it should be noted that, while exemplified herein primarily by way of single needle implementations, the present invention may be implemented with two or more needles. By way of example, FIG. 3 shows an intradermal mini-needle interface, generally designated 30, according to an alternative embodiment of the present invention. Interface 30 is generally structurally and functionally similar to interface 20, differing only in that it provides two channels each receiving a corresponding needle 12. Similar variations, and variations employing more than two needles, may be based on the other embodiments described herein.

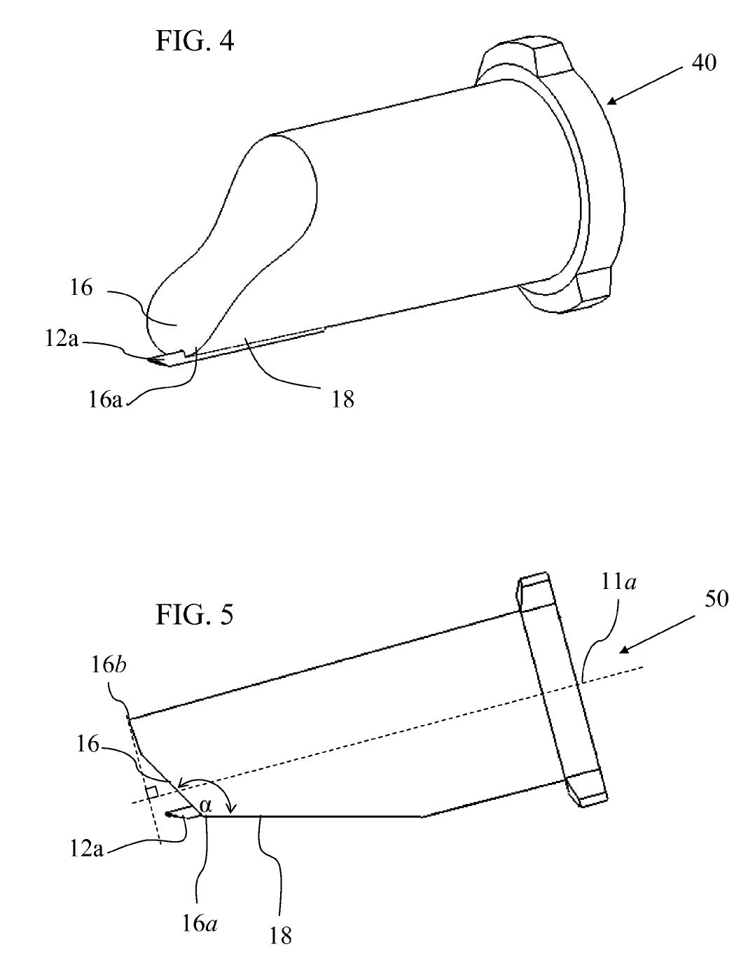

Turning now to FIG. 4, although the present invention is exemplified herein primarily with reference to structures employing flat skin contact surfaces, it should be noted that both penetration limiter 16 and skin contact surfaces 18 may be implemented in a wide range of geometrical forms. By way of one non-limiting example, FIG. 4 shows an intradermal mini-needle interface, generally designated 40, according to an alternative embodiment of the present invention. Interface 40 is generally structurally and functionally similar to interface 20, but employs a rounded skin contact surface 18 and profiled penetration limiter 16.

Parenthetically, interface 40 also exemplifies an implementation in which both the needle axis and the skin contact plane are substantially parallel to the inlet axis. In practice, a small deviation is preferably provided between the needle axis and the skin contact plane in order to ensure that the rear end of the needle is sufficiently deep within the block of the adapter to facilitate a leak-free seal to the internal channel connecting with the inlet.

Turning now to FIG. 5, there is shown an intradermal mini-needle interface, generally designated 50, according to an alternative embodiment of the present invention. Interface 50 is generally structurally and functionally similar to interface 10, and similar reference numerals are used for similar elements. Interface 50 differs from interface 10 primarily in that it has a forward projection 16b, in this case formed as part of penetration limiter 16, which extends forward along the inlet axial direction at least as far as penetrating tip 12a, but upwardly spaced therefrom. As a result, a user is intuitively induced to use the device in the intended shallow-angle insertion mode, since perpendicular approach of the needle to the skin is blocked by the forward projection. Furthermore, this structure provides some degree of protection against inadvertent needle penetration or damage to the needle tip through inadvertent axial impact on the skin or other surfaces.

In the implementation illustrated here, forward projection 16b is generated by providing an obtuse angle α at the intersection of forward penetration limiting surface 16 and skin contact surface 18. In the example shown, an internal angle of about 135 degrees was chosen.

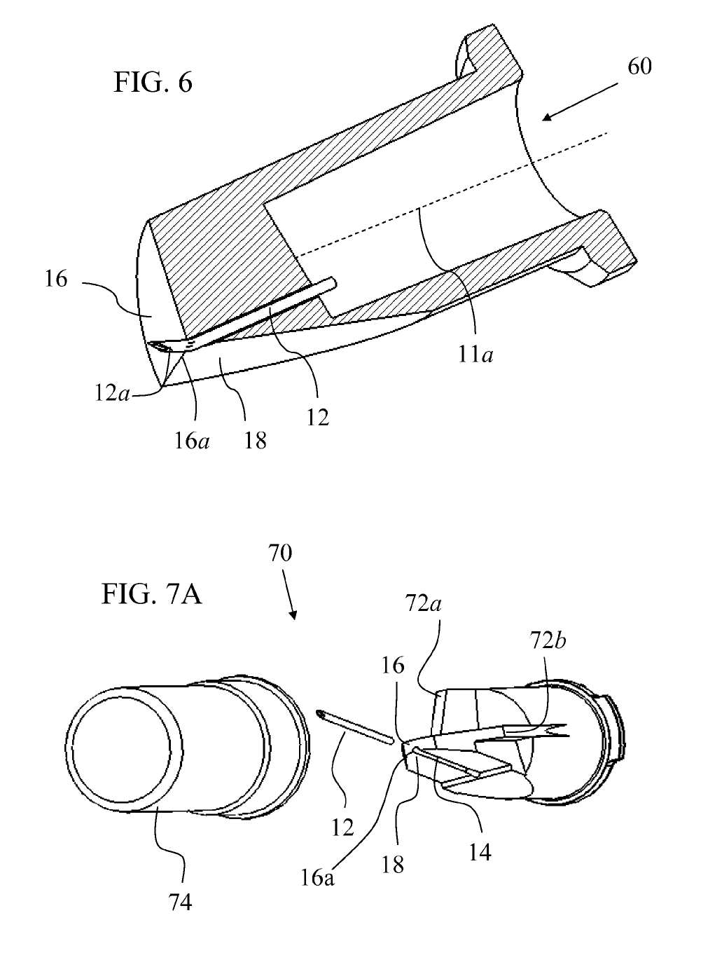

Turning now to FIG. 6, there is shown an intradermal mini-needle interface, generally designated 60, according to an alternative embodiment of the present invention. Interface 60 is generally structurally and functionally similar to interface 10, and similar reference numerals are used for similar elements. Interface 60 differs from interface 10 primarily in that it employs a needle 12 in which penetrating portion 12a is bent at an angle relative to the remainder of the needle. In certain cases, this structure is believed to facilitate engagement between needle and biological barrier. The bent form of the needle may also allow the rear portion of the needle to be arranged roughly parallel to the inlet axis 11a, which may be advantageous in some cases.

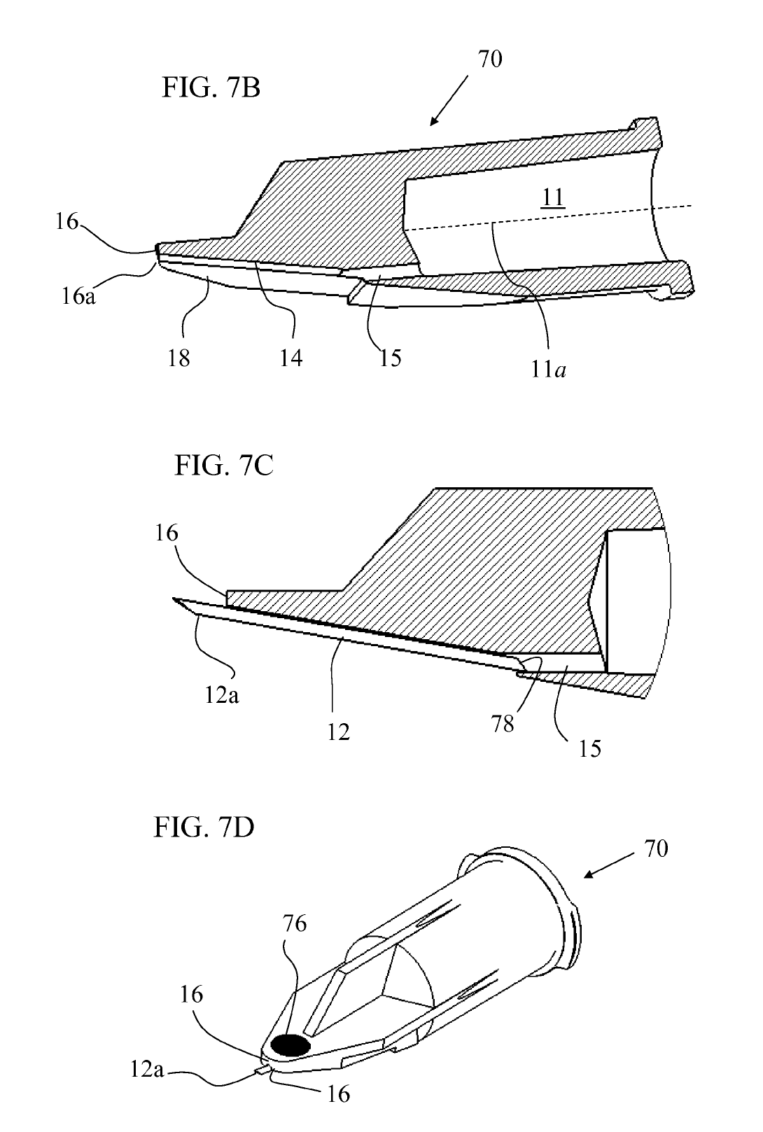

Turning now to FIGS. 7A-7D, there is shown an intradermal mini-needle interface, generally designated 70, according to an alternative embodiment of the present invention. Interface 70 is generally structurally and functionally equivalent to interface 20, and similar reference numerals are used for similar elements, and has features suited for efficient production by injection molding. Specifically, in place of the thick block of the previous embodiments, interface 70 is here implemented with a reduced volume block, referred to as a hub, with thin walls, tapered structures and no undercuts. The device is provided with a cap 74 which protects the needle tip prior to use, sitting securely over ribs 72a and 72b of the hub as a press-fit. The fit is designed to allow pathways for the entry of gas during sterilization (for example, when using EtO). Gamma irradiation and additional sterilization techniques known in the art are also possible.

As best seen in FIGS. 7B and 7C, the flow path is asymmetric relative to axis 11a, and includes an open channel 14 for receiving needle 12 and a closed sleeve 15 connecting between the rear end of needle 12 and inlet 11. As seen in FIG. 7C, the rear end 78 of needle 12 may optionally be beveled as shown, providing a smoother flow path than would otherwise be possible and facilitating good adhesive sealing.

As seen in FIG. 7D, the interface may optionally be provided with an orientation marker 76. The marker in the example is placed on the front top side of the device to indicate to the user which way the device should be held during use. Clearly the marker can be implemented in many ways, including but not limited to, a printed marking, a sticker, a molded structural feature and combinations of the above.

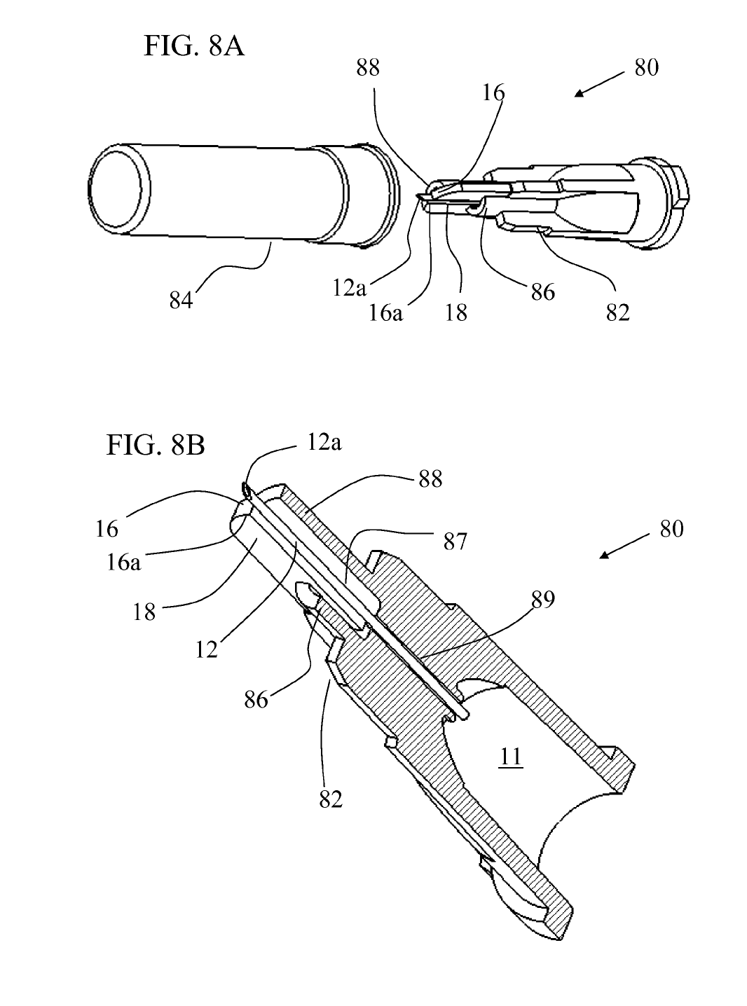

Turning now to FIGS. 8A and 8B, there is shown an intradermal mini-needle interface, generally designated 80, according to an alternative embodiment of the present invention. Interface 80 is generally structurally and functionally similar to interface 70, and similar reference numerals are used for similar elements. In this case, needle 12 is deployed with its central axis generally aligned with the axis of the inlet, and the required geometry of penetration limiter 16 and skin contact surfaces 18 is provided by various structures suited for efficient production by injection molding.

Specifically, penetration limiter 16 is provided by the front surface of a needle leader structure 88 including flattened portions on either side of the needle and a top bridging element for support. Skin contact surfaces 18 are provided by the underside of the needle leader structure 88 as well as by a lower bridging element 86 and stepped lower ribs 82. These surfaces in combination define a shallow angle skin contact plane relative to the needle axis, providing functionality similar to the other embodiments discussed above. The ribs also provide press-fitting of a protective cap 84, as in the previous embodiment.

In addition to providing mechanical support, the upper and lower bridging elements define a recess 87 around the needle, thereby facilitating bonding of the needle and hub through a single-drop bonding procedure. The rear part of needle 12 runs through a closed channel 89 to connect with the inlet 11.

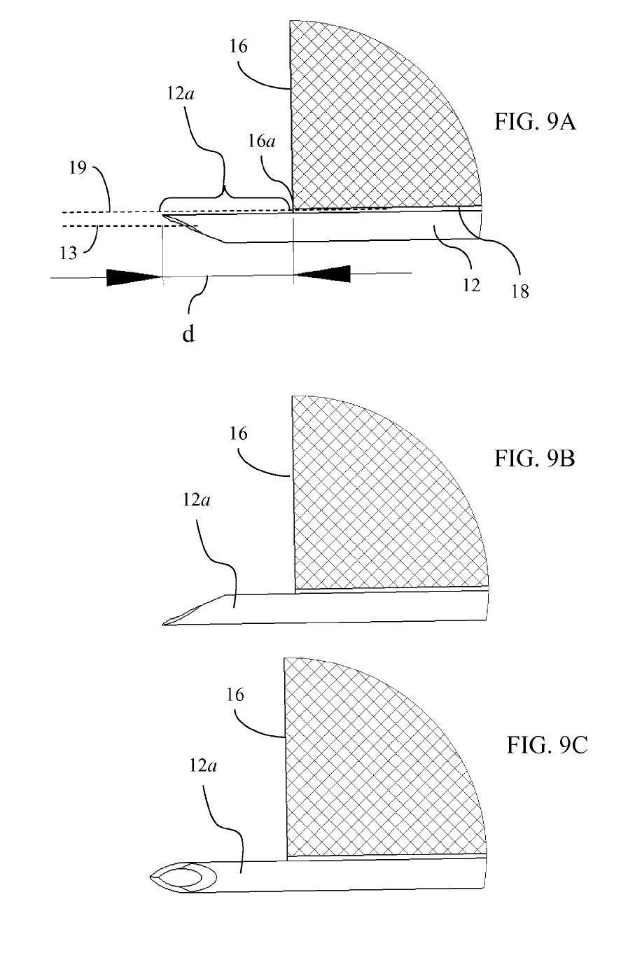

Turning now to FIGS. 9A-9C, it will be noted that the directional skin contact surfaces of embodiments of the present invention provides a well defined orientation of the bevel of penetrating tip 12a relative to the skin. This allows choice of an optimal bevel orientation for a given application. Specifically, the direction of the bevel has been found to impart slight directionality to liquid delivery flow, such that the downward-facing bevel of FIG. 9A delivers fluid to slightly deeper layers of the skin than the upward-facing bevel of FIG. 9B. The sideways-facing bevel of FIG. 9C is believed to reduce the flow impedance by relieving the contact pressure between the outlet aperture and stretched skin layers.

Turning now to FIGS. 10A-10E, it should be noted that a wide range of penetration limiter forms may be used to implement embodiments of the interface of the present invention. The forms illustrated here are non-limiting examples of the penetration limiter 16 as viewed along the needle axis from the distal end, and show the asymmetric positioning of penetrating tip 12a in each example.

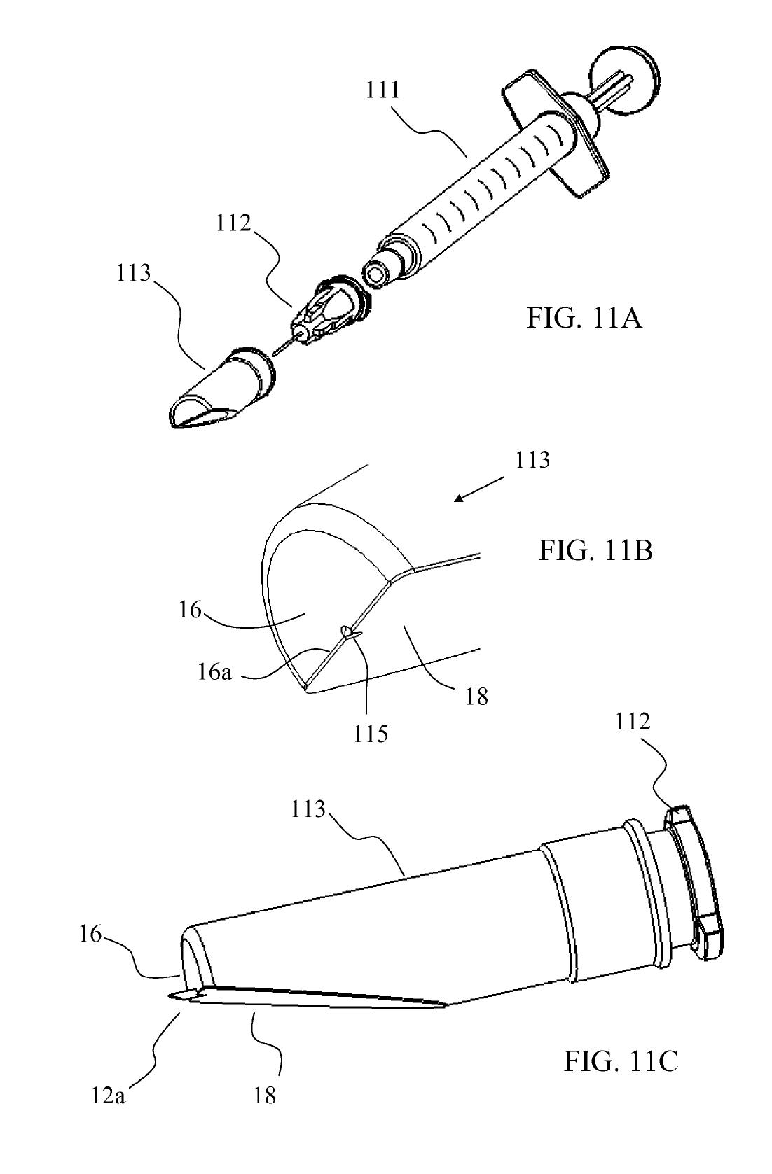

Embodiments of the interface of the present invention have been illustrated thus far with reference to adapter implementations suitable for attachment to a syringe or other standard device for liquid delivery or sampling. It should be noted however that the interface of the present invention is not limited to this adapter modality, and can be implemented in a wide range of other forms. By way of non-limiting examples, FIGS. 11A-13B present three alternative modalities and form factors. It should be noted that each of these modalities may be implemented with any combination of features from the different interface embodiments described herein, and are not limited to the particular geometries of skin contact surfaces or any other specific features shown.

Turning first to FIGS. 11A-11C, these illustrate an implementation in which an embodiment of an interface according to the present invention is implemented using an add-on supplementary adapter 113 with a conventional hypodermic needle adapter 112. Thus, in this case, hypodermic needle 12 is part of needle adapter 112 which has an exposed needle of length greater than 3 mm, and typically in excess of 10 mm. Penetration limiter 16, edge 16a and skin contact surface 18 are all provided by supplementary adapter 113 which is configured for receiving the exposed needle. The combined structure of adapters 112 and 113 provides an interface as shown in FIG. 11C, which is functionally equivalent to the embodiments for FIGS. 1A-8B, suitable for use with a syringe 111 or other device. It should be noted that, in this as in other embodiments of the interface of the present invention, during insertion of the interface into the skin, the penetrating portion 12a and the various skin contact surfaces (penetration limiter 16, edge 16a and skin contact surface 18) remain in fixed spatial relation to each other.

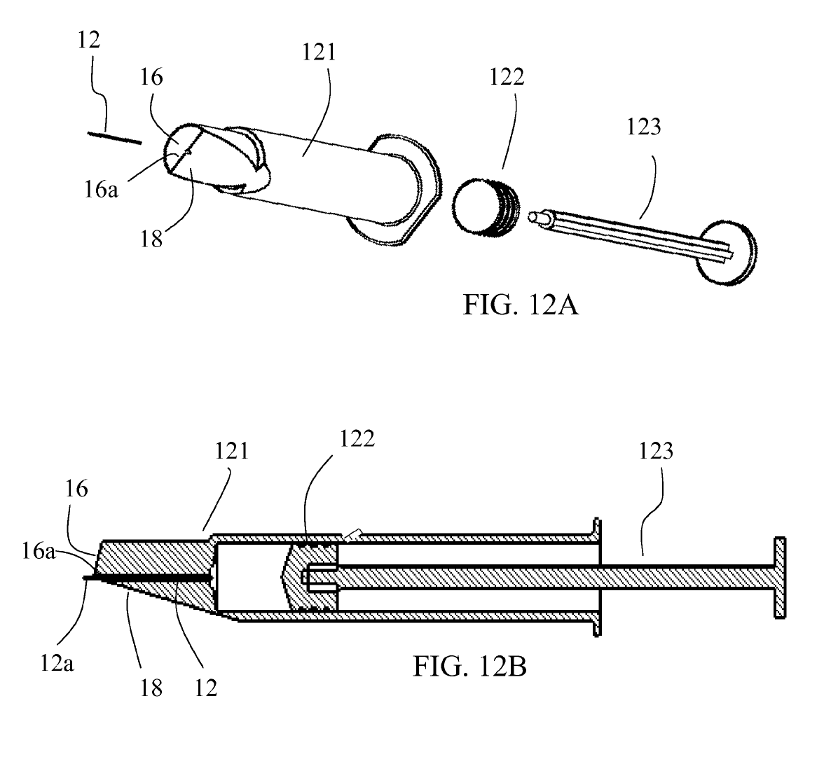

FIGS. 12A and 12B show an implementation in which the intradermal mini-needle interface is integrated as the front end of a syringe body 121 for use with a piston 122 and plunger 123. The structure and function of needle 12, penetrating portion 12a, penetration limiter 16, edge 16a and skin contact surface 18 remain as described in one or more of the previous embodiments. This implementation is particularly suited, although not limited, to use in pre-filled syringe applications.

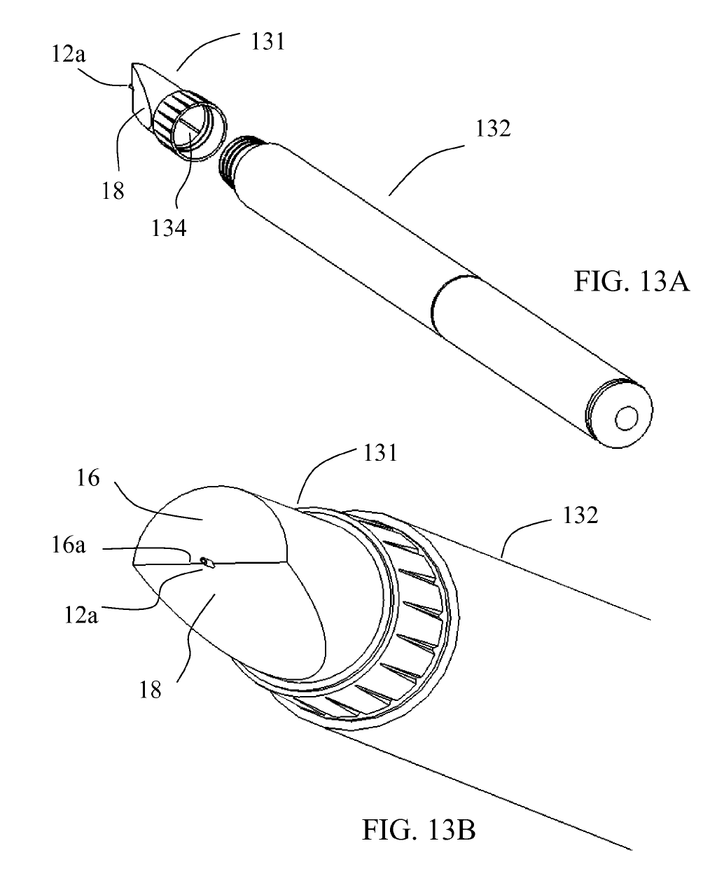

FIGS. 13A and 13B show a further implementation of an interface 131 according to an embodiment of the present invention in which the interface is provided with a rearward pointing septum needle 134 for use with an injector device 132. Optionally, rearward pointing septum needle 134 may be a rearward extending part of the same needle that provides penetrating portion 12a. In other cases, different gauges of needle may be preferred for the skin interface and the septum needle. Here again, the structure and function of penetrating portion 12a, penetration limiter 16, edge 16a and skin contact surface 18 remain as described in one or more of the previous embodiments.

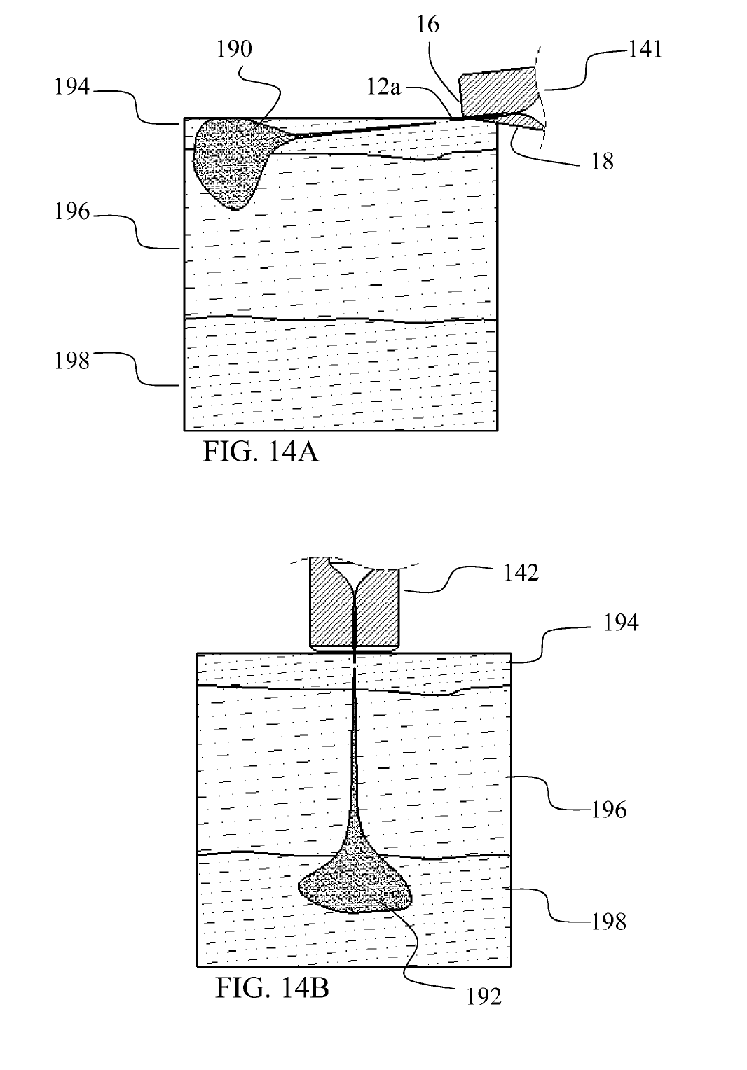

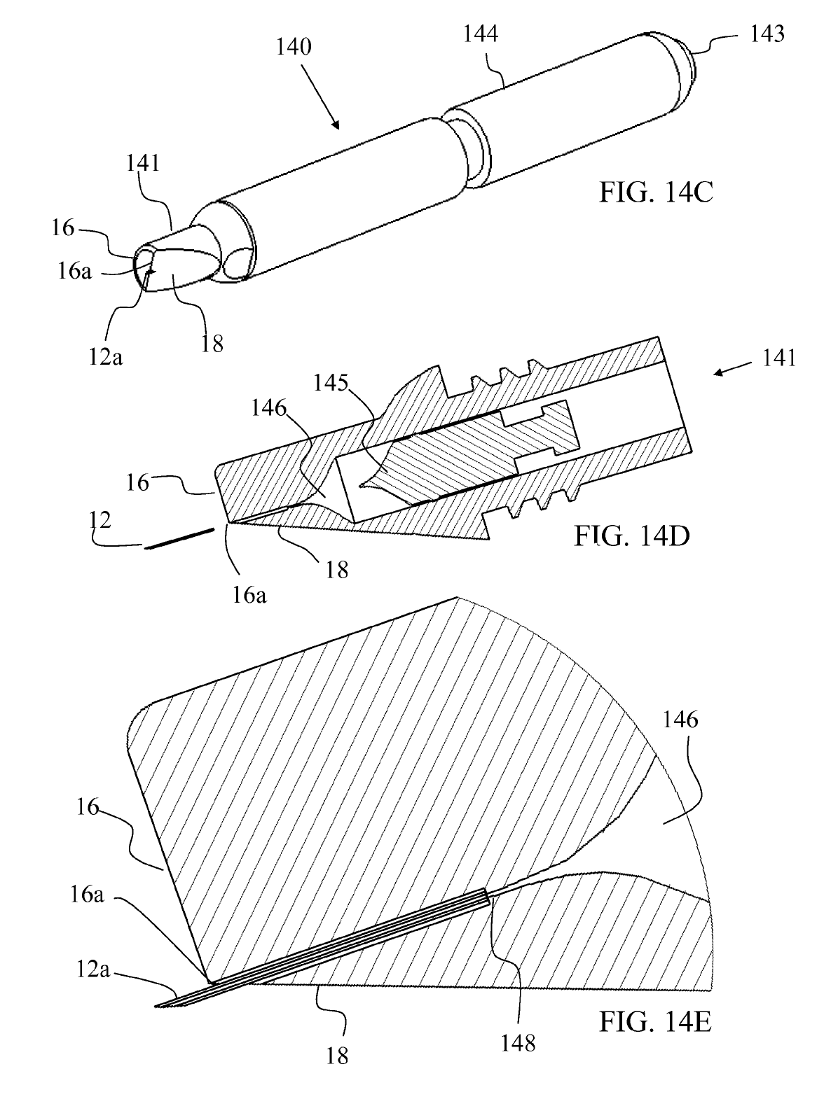

Turning now to FIGS. 14A-14E, a further aspect of the present invention relating to “lateral jet injection” or “intradermal jet injection” will be described. According to this aspect of the invention, after penetrating at least one layer of the skin with a hollow needle, the needle is maintained with its central axis at an angle of less than 45 degrees to the initial local surface of the skin, and a high energy jet of liquid is directed along the hollow needle so as to cause directional penetration of the liquid through tissue beyond an end of the hollow needle.

Jet injectors have been known in the art for many years (for example US Patent Application Pub. No. US 2004/0220524 A1 of Antares Pharma), but are typically designed to be used in a perpendicular approach and thus are able to deliver the medicament only to the deep subcutaneous or the intramuscular compartments.

The disclosed lateral jet injection method sprays a jet laterally into the dermal compartment and therefore can be regarded as a distinctive route of administration. It differs from the ordinary perpendicular jet injection as it targets shallower tissue (ID instead of IM/SC), and it differs from ordinary ID injection as it disposed in the skin as a dispersed spray rather than as a bleb reservoir.

It is a particularly preferred feature of certain implementations of the present invention that the high pressure flow is delivered only after mechanical penetration by penetrating portion 12a, thereby ensuring that the material of the jet successfully penetrates beneath the stratum corneum. In the absence of such initial mechanical penetration, it is believed that use of a shallow angle jet would result in a significant proportion of the material in the jet being deflected outwards by the stratum corneum and lost, reducing efficiency and rendering results of the procedure unpredictable. Pre-penetration of the stratum corneum also allows lower jet pressures to be used to achieve a given desired degree of penetration.

The lateral intradermal jet injection route is assumed to have benefits of enhancing the pharmacokinetics parameters of certain drugs and treatments. One example is a possible improved absorption of insulin as the spray is dispersed over large areas of the papillary dermis. Another example is a possibly improved immune response in vaccines as the jet transverse through regions rich in langerhance cells.

Another benefit of Intradermal Jet Injection concerns the reduced injection time (fraction of second) compared to regular intradermal injection (about 5-10 seconds).

FIGS. 14A and 14B are illustrative comparative views of a lateral intradermal jet injection according to the present invention and a conventional perpendicular jet injection (prior art), respectively.

An exemplary device for performing the lateral jet injection technique according to the present invention is shown in FIG. 14C, with an interface 141 attached to a jet injector device 140 with a pulsed pressure device 144 operated by a trigger button 143. Other than interface 141, the structure and function of jet injector device 140 are equivalent to those of commercially available jet injectors, and will not be described here in detail. FIGS. 14D and 14E show enlarged views of interface 141 which preferably maintains the structural features of the interfaces described above (and similarly numbered) while providing a modified flow path and reduced internal diameter suitable for delivering the high energy jet. Specifically, interface 141 includes a tapered inlet cavity 146 tapering to a nozzle 148 of diameter matched to the internal diameter of the needle. The pulsed pressure acts on a correspondingly formed piston 145 to generate the high energy jet.

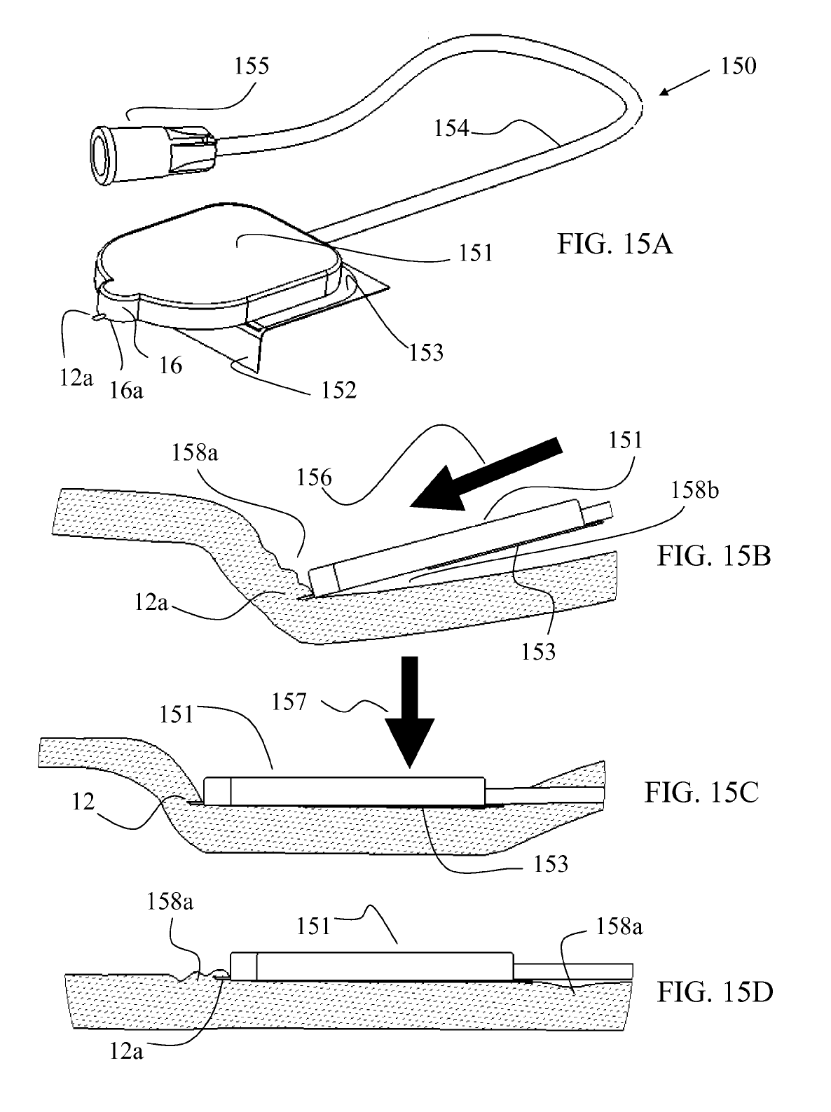

Turning now to FIGS. 15A-15D, these illustrates a further implementation of the interface of the present invention providing an infusion set. In this case, penetration limiter 16, edge 16a, skin contact surface (not shown) and penetrating portion 12a are implemented on one side of a flat patch-like device 151 from which extends a length of tubing 154 terminating in a connector 155 to provide a fluid flow path to the needle. Part of the underside of device 151 is provided with a layer of adhesive 153 covered by peel-off backing 152.

FIGS. 15B-15D illustrate the deployment of device 151 and, by analogy, of the other interfaces of the present invention described above. The device is first brought in contact with the skin at a shallow angle and moved laterally, in the forward direction of the needle, while in contact with the skin. This motion stretches the skin under the device 158b while reducing tension in the skin ahead of the needle 158a, typically also forming a nick of skin into which the needle penetrates. It will be noted that edge 16a generally delineates the boundary between stretched region 158b and unstretched region 158a of skin, and the needle is inserted into the unstretched region.

In the case of an infusion set, the insertion process preferably includes additional steps as illustrated in FIGS. 15C and 15D. Specifically, device 151 is then pressed flat against the skin surface (FIG. 15C) while maintaining the sideways insertion force so as to make the device adhere to the skin. This maintains the portion of skin under the device stretched, and regions 158a unstretched/relaxed, even after manual pressure is released. The final deployed state of the device is illustrated in FIG. 15D.

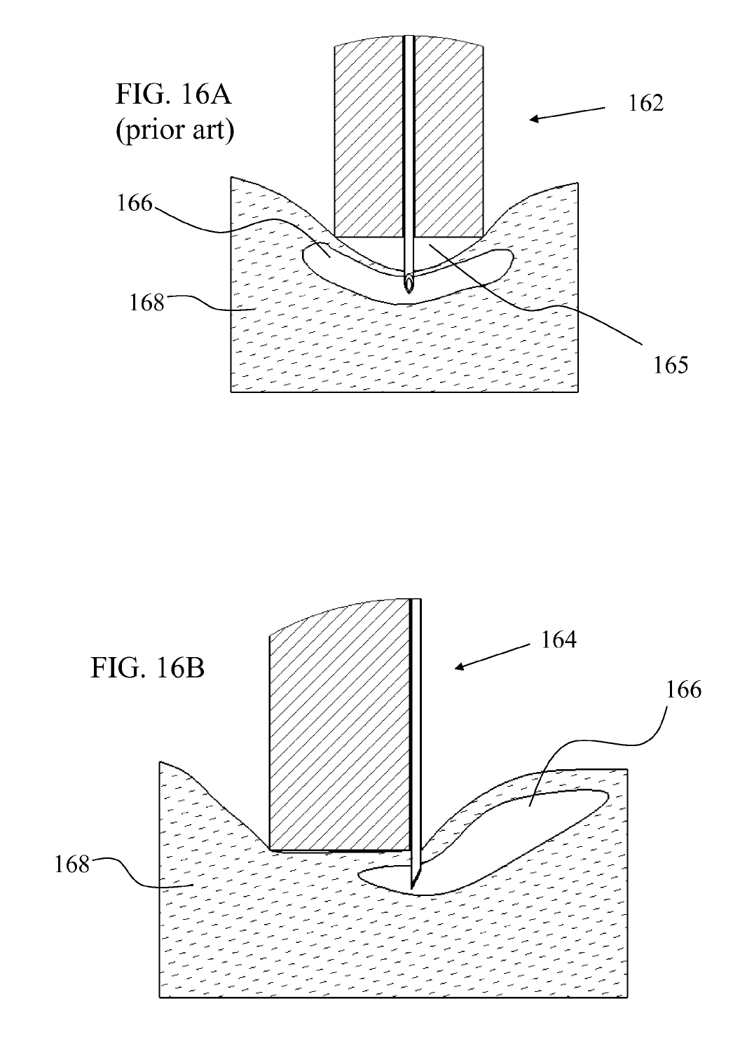

Turning now to FIGS. 16A and 16B, although described herein for use in shallow angle intradermal injection, it should be noted that the device structures described herein may exhibit significant advantages even if used for perpendicular approach. Specifically, it is notable that in a conventional symmetrical mini-needle device 162 (FIG. 16A), the skin is typically deformed around the needle, causing reduced penetration. Additionally, the site of the bleb formation 166 within the skin 168 is overlaid by the penetration limiter 165 and compressed by its downward pressure, thereby increasing the force that is required to inject the fluid. In contrast, the asymmetrical penetration limiter 164 of FIG. 16B has the needle deployed on the edge of the block, preventing the skin 168 from deforming around the needle. Penetration is therefore more consistent and reliable. Furthermore, the site of the bleb 166 is not directly compressed by the limiter arrangement (especially in the case as shown where the bevel is directed outwards from the configuration, i.e., what it referred to as “bevel down” in the shallow angle insertion mode of use) so the force required for injection is reduced.

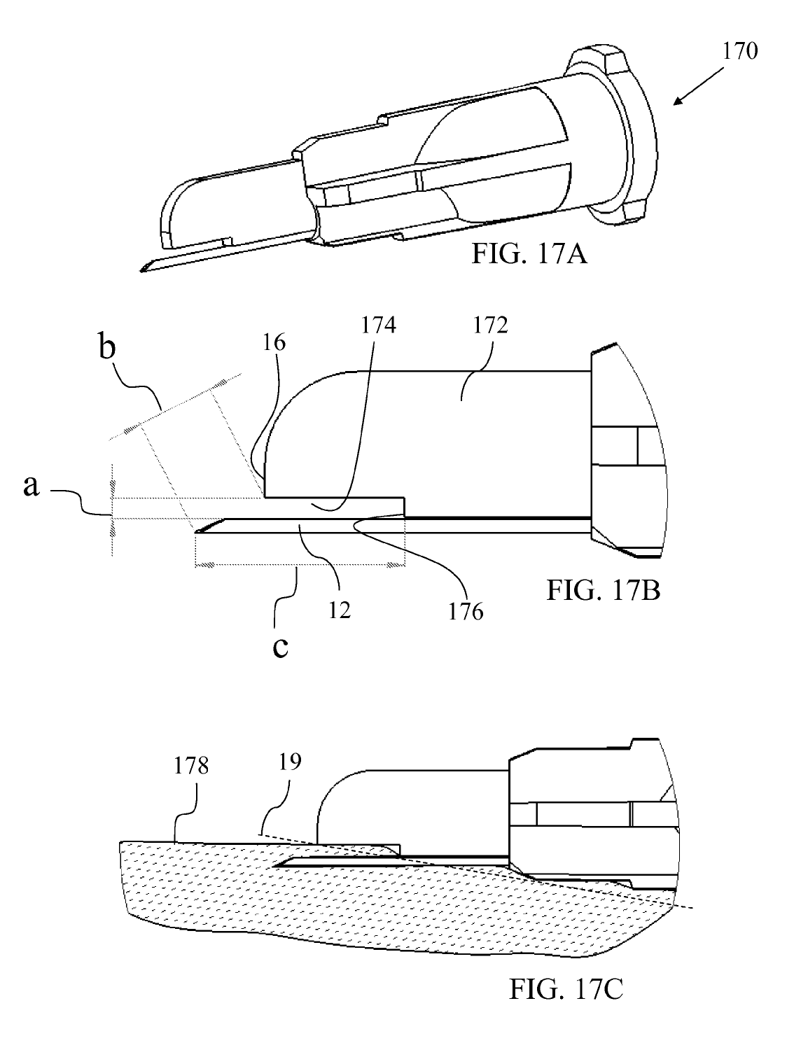

Finally, turning to FIGS. 17A-17C, there is shown a further alternative embodiment of an intradermal mini-needle interface, generally designated 170, according to an alternative embodiment of the present invention. Interface 170 is generally structurally and functionally similar to the interfaces described above, and similar reference numerals are used for similar elements.

Interface 170 provides its penetration limiter through a projecting element 172 which extends along one side of hypodermic needle 12 and defines a maximum depth of forward penetration b no greater than 3 mm, as in the previous examples. Unlike the previous examples, the projecting element 172 is spaced from the needle along part of the length of the needle. Specifically, projecting element 172 has an edge 174 facing, but spaced from, the needle by a spacing a no greater than 2 mm, and more preferably, between about 0.5 mm and about 1.5 mm. This arrangement allows extended penetration of the needle within the layers of the skin as illustrated in FIG. 17C, while limiting the depth of penetration beneath the skin surface 178. Edge 174 terminates at a step 176 where the gap between projecting element 172 and the needle is closed, defining a maximum length c of intradermal penetration.

Notwithstanding the description of FIG. 16B above, it should be noted that a preferred mode of use, and corresponding method, according to embodiments of the present invention includes penetrating at least one layer of the skin with the hypodermic needle and, at the same time or subsequent thereto, bringing the hypodermic needle to a position with its central axis at a shallow angle to the initial local surface of the skin such that the hypodermic needle extends into a portion of the skin which is not overlaid by the penetration limiter. A liquid is then transferred via the hypodermic needle through at least one layer of the skin.

It will be appreciated that the above descriptions are intended only to serve as examples, and that many other embodiments are possible within the scope of the present invention as defined in the appended claims.

What is claimed is:

1. An intradermal mini-needle interface comprising:
(a) a penetration limiter providing a skin contact edge;
(b) a hollow hypodermic needle having a beveled penetrating portion protruding forward beyond said skin contact edge by no more than 3 mm, said penetrating portion having a central needle axis; and
(c) at least one skin contact surface defining a skin contact plane parallel to, or at a shallow angle to, said needle axis, said skin contact plane intersecting said skin contact edge substantially at a base of said penetrating portion.

2. The intradermal mini-needle interface of claim 1, wherein said penetration limiter is asymmetric under rotation about said needle axis.

3. The intradermal mini-needle interface of claim 1, wherein said penetration limiter is non-encompassing relative to said needle axis.

4. The intradermal mini-needle interface of claim 1, wherein said penetration limiter is provided by a projecting element extending along one side of said hypodermic needle and spaced therefrom by no more than 2 mm.

5. The intradermal mini-needle interface of claim 1, further comprising a fluid inlet in fluid communication with said hypodermic needle, said fluid inlet defining an inlet axis, wherein said skin contact plane is at an oblique angle to said inlet axis.

6. The intradermal mini-needle interface of claim 5, wherein said needle axis is non-parallel to said inlet axis.

7. The intradermal mini-needle interface of claim 1, wherein said hypodermic needle is deployed substantially parallel to, and adjacent to, said skin contact plane.

8. The intradermal mini-needle interface of claim 1, wherein said penetration limiter and said at least one skin contact surface are provided by a unitary block of polymer material.

9. The intradermal mini-needle interface of claim 1, wherein said hypodermic needle is part of a needle adapter having an exposed needle of length greater than 3 mm, and wherein said penetration limiter and said at least one skin contact surface are provided by a supplementary adapter configured for receiving said exposed needle.

10. The intradermal mini-needle interface of claim 1, wherein the intradermal mini-needle interface is integrated as part of a syringe body.

11. The intradermal mini-needle interface of claim 1, further comprising a fluid inlet in fluid communication with said hypodermic needle, said fluid inlet being configured for removable attachment to a syringe.

12. The intradermal mini-needle interface of claim 1, further comprising a pressure impulse supply arrangement associated with said hypodermic needle and deployed to deliver a high energy fluid jet through said hypodermic needle.

13. A method for transferring a liquid through at least one layer of the skin by use of an intradermal mini-needle interface including:
(a) a penetration limiter providing a skin contact edge;
(b) a hypodermic needle having a beveled penetrating portion protruding forward beyond said skin contact edge by no more than 3 mm, said penetrating portion having a central needle axis; and
(c) at least one skin contact surface defining a skin contact plane parallel to, or at a shallow angle to, said needle axis, said skin contact plane intersecting said skin contact edge substantially at a base of said penetrating portion,
the method comprising the steps of:
(i) penetrating at least one layer of the skin with the hypodermic needle;
(ii) bringing the hypodermic needle to a position with its central axis at a shallow angle to the initial local surface of the skin such that the hypodermic needle extends into a portion of the skin which is not overlaid by the penetration limiter; and
(iii) transferring a liquid via the hypodermic needle through at least one layer of the skin.

14. A method for introducing a liquid through at least one layer of the skin, the method comprising the steps of:
(a) penetrating at least one layer of the skin with a hollow needle;
(b) maintaining the hollow needle with its central axis at an angle of less than 45 degrees to the initial local surface of the skin; and
(c) directing a high energy jet of the liquid along the hollow needle so as to cause directional penetration of the liquid through tissue beyond an end of the hollow needle.

15. The method of claim 14, wherein the hollow needle is part of an intradermal mini-needle interface comprising:
(a) a penetration limiter providing a skin contact edge;
(b) a hypodermic needle having a beveled penetrating portion protruding forward beyond said skin contact edge by no more than 3 mm, said penetrating portion having a central needle axis; and
(c) at least one skin contact surface defining a skin contact plane parallel to, or at a shallow angle to, said needle axis, said skin contact plane intersecting said skin contact edge substantially at a base of said penetrating portion.
